(19) United States
(12) Patent Application Publication
(10) Pub. No.: US 2007/0248560 A1
(43) Pub. Date: Oct. 25, 2007

(54) Cosmetic product comprising at least one cyanoacrylate monomer and at least one non-ionic polyurethane

(76) Inventors: Aude Livoreil, Paris (FR); Gaelle Brun, Paris (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/785,005

(22) Filed: Apr. 13, 2007

Related U.S. Application Data
(60) Provisional application No. 60/810,154, filed on Jun. 2, 2006.

(30) Foreign Application Priority Data
Apr. 13, 2006 (FR) 06/03286

(51) Int. Cl. A61K 8/81; A61K 8/73; C08L 83/08

(52) U.S. Cl. 424/70.16; 424/70.17; 525/440.03

(57) ABSTRACT

Disclosed herein is a cosmetic product for treating keratin fibers, for example, human keratin fibers such as the hair, comprising at least one polymerizable cyanoacrylate monomer, and at least one non-ionic polyurethane, wherein the at least one polymerizable cyanoacrylate monomer and the at least one non-ionic polyurethane may be present in the same composition or in separate form, and may be applied, at the time of use, either together, separately, simultaneously, or sequentially over the time. Further disclosed herein is a cosmetic composition comprising at least one polymerizable cyanoacrylate monomer and at least one non-ionic polyurethane. Still further disclosed herein are methods for treating, dyeing, and/or conditioning keratin fibers comprising applying to the fibers at least one composition of the present disclosure.

This application claims benefit of U.S. Provisional Application No. 60/810,154, filed Jun. 2, 2006, the contents of which are incorporated herein by reference. This application also claims benefit of priority under 35 U.S.C. §119 to French Patent Application No. FR 06/03286, filed April 13, 2006, the contents of which are also incorporated herein by reference.

Disclosed herein is a cosmetic product for treating keratin fibers, for example, human keratin fibers such as the hair, comprising at least one cyanoacrylate monomer and at least one non-ionic polyurethane.

The present disclosure further relates to an embodiment of the cosmetic product provided in the form of a composition, and to a method for treating the hair comprising applying the composition to the hair.

The hair is generally damaged and embrittled by the action of external atmospheric agents such as light and adverse weather conditions, and by mechanical or chemical treatments such as brushing, combing, bleaching, permanent waving and/or dyeing. The result is that the hair is often difficult to manage, for instance, it may be difficult to disentangle or style, and the hair, even when lush, may have difficulty preserving a hairstyle of good appearance because of the fact that the hair lacks vitality, volume, and/or liveliness.

It is now customary to use hair-styling products which make it possible to condition the hair by improving its body, mass, and/or volume.

These hair-styling products are generally cosmetic hair compositions comprising at least one polymer which exhibits high affinity for the hair and which may have the role of forming a film at its surface in order to modify the surface properties of the hair, for example, to condition the hair.

One disadvantage linked to the use of these hair compositions lies in the fact that the cosmetic effects conferred by such compositions tend to disappear, for instance, after the first shampoo.

It is possible to overcome this disadvantage by increasing the stability of the polymer deposit by directly carrying out a free-radical polymerization of certain monomers on the hair.

However, the treatments thus obtained may be unacceptable from the cosmetic point of view. Indeed, a high degradation of the fiber probably related to the presence of polymerization initiators is generally observed and the hair thus treated may be difficult to disentangle.

It is known in the art, for example, in French Patent No.2 833 489, to use anionic-polymerizing cyanoacrylate monomers directly at the surface of the hair in the presence of a nucleophilic agent such as hydroxide ions (OH⁻) contained in water at neutral pH. Thus, once deposited on the hair, these monomers form a polymer leading to conditioning which is stable to shampoos.

However, it has been observed that the cosmetic properties obtained from this method are not satisfactory.

It is therefore desirable to provide cosmetic compositions, for example, compositions for conditioning the hair, which are stable to shampoos while preserving good cosmetic properties, i.e., compositions that durably provide body, mass, and/or volume to the hair.

The present inventors have discovered that the combination of at least one non-ionic polyurethane and at least one polymerizable cyanoacrylate monomer may overcome at least one of the disadvantages discussed above.

The present inventors have also observed that on applying such a composition to the hair, a stable covering or coating is formed in situ. Without wishing to be bound by theory, it appears that the hydroxide ions (OH⁻) contained in water at neutral pH absorbed by the hair set in motion the anionic polymerization process at the treatment composition-hair interface. The polyurethane present in the composition thus becomes trapped in the polymer structure obtained, which makes it possible to improve the cosmetic properties of the hair.

Thus, the compositions disclosed herein may make it possible to durably improve the cosmetic properties of the hair.

Disclosed herein is thus a cosmetic product for treating keratin fibers, for example, human keratin fibers such as the hair, comprising:

at least one polymerizable cyanoacrylate monomer, and

at least one non-ionic polyurethane,

wherein the at least one polymerizable cyanoacrylate monomer and the at least one non-ionic polyurethane are present in the same composition or in separate form, and

wherein the at least one polymerizable cyanoacrylate monomer and the at least one non-ionic polyurethane may be applied, at the time of use, either together or separately, i.e., simultaneously or sequentially over time.

In at least one embodiment, the cosmetic product for treating keratin fibers, such as the hair, comprises the at least one polymerizable cyanoacrylate monomer and the at least one non-ionic polyurethane present in the same composition.

Also disclosed herein is a method for cosmetic treatment comprising applying a cosmetic composition of the present disclosure to the hair. In at least one embodiment, the cosmetic treatment is a conditioning treatment for the hair.

Further disclosed herein is a method for dyeing the hair comprising applying the cosmetic composition of the present disclosure to the hair.

Still further disclosed herein is a multi-compartment device or kit comprising at least one compartment containing a composition according to the present disclosure, and at least one second compartment comprising at least one nucleophilic agent.

Other subjects, characteristics, aspects and advantages of the present disclosure will emerge more clearly upon reading the description and the examples which follow.

Cyanoacrylate Monomers

The at least one cyanoacrylate monomer present in the composition of the present disclosure may be chosen, by non-limiting example, from monomers of formula (I):

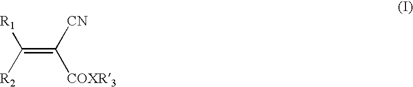

wherein:

X is chosen from NH, S, and O,

R₁ and R₂, which may be identical or different, are chosen from weakly or non-electron-attracting (weakly or non-inductive-attracting) groups such as:
hydrogen,
saturated or unsaturated, linear, branched, or cyclic hydrocarbon groups comprising, for example, from 1 to 20, or from 1 to 10 carbon atoms, and optionally comprising at least one atom chosen from nitrogen, oxygen, and sulphur atoms, and optionally substituted with at least one group chosen from —OR, —COOR, —COR, —SH, —SR, —OH, and halogen atoms,
modified or unmodified polyorganosiloxane residues, and
polyoxyalkylene groups; and

R is chosen from saturated or unsaturated linear, branched or cyclic hydrocarbon groups comprising, for example, from 1 to 20, or from 1 to 10 carbon atoms, and optionally comprising at least one atom chosen from nitrogen, oxygen, and sulphur atoms, and optionally substituted with at least one group chosen from —OR′, —COOR′, —COR′, —SH, —SR′, —OH, halogen atoms, and polymer residues which may be obtained by a process chosen from free-radical polymerization, polycondensation, and ring opening, wherein R′ is chosen from C₁-C₁₀ alkyl groups; and

R′₃ is chosen from hydrogen, saturated or unsaturated, linear, branched, or cyclic hydrocarbon groups comprising, for example, from 1 to 20, or from 1 to 10 carbon atoms, and optionally comprising at least one atom chosen from nitrogen, oxygen, and sulphur atoms, and optionally substituted with at least one group chosen from —OR′, —COOR′, —COR′, —SH, —SR′, —OH, halogen atoms, and polymer residues which may be obtained by a process chosen from free-radical polymerization, polycondensation, and ring opening, wherein R′ is chosen from C₁-C₁₀ alkyl groups.

As used herein, the expression “electron-attracting or inductive-attracting group” is understood to mean any group which is more electronegative than carbon. Such groups are described, for example, in PR Wells Prog. Phys. Org. Chem., Vol. 6, p. 111 (1968).

As used herein, the expression “weakly or non-electron-attracting group” is understood to mean any group whose electronegativity is less than or equal to that of carbon.

According to one embodiment, the alkenyl or alkynyl groups disclosed above may comprise from 2 to 20 carbon atoms, for example, from 2 to 10 carbon atoms.

With regard to the saturated or unsaturated, linear, branched, or cyclic hydrocarbon groups, non-limiting examples include C₁-C₂₀ linear or branched alkyl, alkenyl, and alkynyl groups such as methyl, ethyl, n-butyl, tert-butyl, isobutyl, pentyl, hexyl, octyl, butenyl, butynyl; cycloalkyl, and aromatic groups.

Non-limiting examples of substituted hydrocarbon groups include hydroxyalkyl and polyhaloalkyl groups.

With regard to the modified or unmodified polyorganosiloxane residues, examples of unmodified polyorganosiloxanes include, but are not limited to, polyalkylsiloxanes such as polydimethylsiloxanes, polyarylsiloxanes such as polyphenylsiloxanes, and polyarylalkylsiloxanes such as polymethylphenylsiloxanes. Suitable modified polyorganosiloxanes include, for example, polydimethylsiloxanes comprising at least one group chosen from polyoxyalkylene, siloxy, silanol, amine, imine, and/or fluoroalkyl groups.

With respect to the polyoxyalkylene groups, non-limiting examples of polyoxyalkylene groups include polyoxyethylene groups and polyoxypropylene groups, and in at least one embodiment, include polyoxyethylene groups and polyoxypropylene groups comprising from 1 to 200 oxyalkylenated units.

Examples of mono- and polyfluoroalkyl groups disclosed above include, but are not limited to, —(CH₂)ₙ—(CF₂)ₘ—CF₃ and —(CH₂)ₙ—(CF₂)ₘ—CHF₂ groups wherein n is an integer ranging from 1 to 20 and m is an integer ranging from 1 to 20.

In at least one embodiment, the substituents R₁ and R₂ may be optionally substituted with a group having a cosmetic activity. Examples of such cosmetic activities include, but are not limited to, those obtained from groups having at least one function chosen from dyeing, antioxidant, UV-screening, and conditioning functions.

Groups having a dyeing function may include, for example, azo, quinone, methine, cyanomethine, and triarylmethane groups.

Groups having an antioxidant function may include, for instance, butylated hydroxyanisole (BHA), butylated hydroxytoluene (BHT), and vitamin E groups.

Groups having a UV-screening function may include, for example, benzo-phenone, cinnamate, benzoate, benzylidenecamphor, and dibenzoylmethane groups.

Groups having a conditioning function may include, for instance, cationic groups of the fatty ester type.

According to one embodiment, R₁ and R₂ are both hydrogen.

In at least one embodiment, R′₃ is chosen from saturated hydrocarbon groups comprising from 1 to 10 carbon atoms and C₂-C₁₀ alkenyl groups.

According to another embodiment, X is O.

Non-limiting examples of compounds of formula (I) include the monomers:

a) belonging to the polyfluoroalkyl 2-cyanoacrylate family such as:

the 2,2,3,3-tetrafluoropropyl ester of 2-cyano-2-propenoic acid of formula (II):

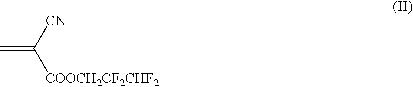

and the 2,2,2-trifluoroethyl ester of 2-cyano-2-propenoic acid of formula (III):

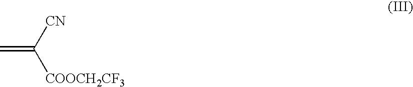

b) belonging to the alkyl and alkoxyalkyl 2-cyanoacrylates of formula (IV):

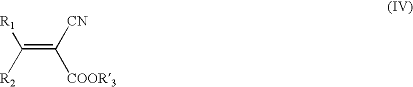

wherein R′₃ is chosen from C₁-C₁₀ alkyl radicals and (C₁-C₄)alkoxy(C₁-C₁₀)alkyl radicals.

Examples of such compounds include, but are not limited to, ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, allyl 2-cyanoacrylate, 2-methoxypropyl 2-cyanoacrylate, 2-ethoxyethyl, 2-methoxyethyl 2-cyanoacrylate, 2-methylheptyl 2-cyanoacrylate, 2-propoxyethyl 2-cyanoacrylate, n-octyl 2-cyanoacrylate, and isoamyl 2-cyanoacrylate.

In at least one embodiment, the at least one cyanoacrylate monomer is chosen from monomers b), i.e., alkyl and alkoxyalkyl 2-cyanoacrylates of formula (IV). According to a further embodiment, the at least one cyanoacrylate monomer is chosen from C₆-C₁₀ alkyl cyanoacrylates.

In yet another embodiment, the at least one cyanoacrylate monomer is chosen from octyl cyanoacrylates of formula (V) and mixtures thereof:

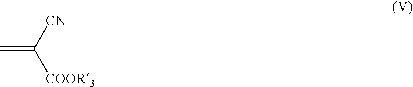

wherein R′₃ is chosen from:
—(CH₂)₇—CH₃,
—CH(CH₃)—(CH₂)₅—CH₃,
—CH₂—CH(C₂H₅)—(CH₂)₃—CH₃,
—(CH₂)₅—CH(CH₃)—CH₃, and
—(CH₂)₄—CH(C₂H₅)—CH₃.

The at least one cyanoacrylate monomer used in accordance with the present disclosure may be covalently linked to at least one support such as polymers, oligomers, and dendrimers. The polymers and oligomers may be chosen from linear, branched, comb, and block polymers and oligomers. The distribution of the at least one cyanoacrylate monomer on the polymeric, oligomeric, or dendritic support may be random, at the terminal position, or in the form of blocks.

In one embodiment of the present disclosure, the cyanoacrylate monomers of formula (I) are monomers capable of anionic polymerization in the presence of at least one nucleophilic agent. As used herein, the expression “anionic polymerization” is understood to mean the mechanism as defined, for example, in “Advanced Organic Chemistry”, Third Edition by Jerry March, pages 151 to 161.

The at least one cyanoacrylate monomer of formula (I) may be synthesized according to methods described in the art. For example, the at least one cyanoacrylate monomer may be synthesized according to the methods described in U.S. Pat. Nos. 3,527,224, 3,591,767, 3,667,472, 3,995,641, 4,035,334, and 4,650,826.

The at least one cyanoacrylate monomer may be present in the cosmetic composition in an amount ranging from 0.1% to 99.9% by weight, for example, from 0.5 to 40% by weight, relative to the total weight of the composition.

Non-Ionic Polyurethanes

The composition according to the present disclosure comprises at least one non-ionic polyurethane, which may be chosen from non-associative and associative polyurethanes.

Non-Associative Polyurethanes

As used herein, the expression “non-associative polyurethane” is understood to mean polycondensates comprising at least one polyurethane block and not comprising, in their structure, a terminal or pendant alkyl or alkenyl chain comprising more than 10 carbon atoms. Such compounds are described, for example, in European Patent Nos. 0 751 162, 0 637 600, 0 648 485, 0 619 111, and 0 656 021, French Patent No. 2 743 297, and International Patent Application Publication No. WO 94/03510.

The non-associative polyurethanes used in accordance with the present disclosure may be soluble in the cosmetically acceptable aqueous medium, or alternatively, may form a dispersion in this medium. In the latter embodiment, the dispersion may then comprise at least 0.05% of at least one surfactant allowing the at least one non-associative polyurethane to be maintained in dispersion.

The at least one surfactant may be any type of surfactant and, in at least one embodiment, a non-ionic surfactant. The mean size of the non-associative polyurethane particles in the dispersion may range, for example, from 0.1 to 1 micrometer.

According to one embodiment of the present disclosure, the at least one non-associative polyurethane may be formed by an arrangement of blocks, this arrangement comprising:

(1) at least one compound comprising two or more active hydrogen atoms per molecule;

(2) at least one diol or a mixture of diols comprising acid functional groups or their salts; and

(3) at least one di- or polyisocyanate.

In at least one embodiment, the compounds (1) may be chosen from diols, diamines, polyesterols, polyetherols, and mixtures thereof.

Examples of compounds (1) include, but are not limited to, linear polyethylene glycols and linear polypropylene glycols, for example, those obtained by the reaction of ethylene or propylene oxide with water or of diethylene or dipropylene glycol in the presence of sodium hydroxide as catalyst. These polyalkylene glycols may have a molecular mass ranging, for instance, from 600 to 20 000.

Other non-limiting examples of compounds (1) include those which comprise at least one group chosen from mercapto, amino, carboxyl, and hydroxyl groups. Such compounds include, for example, polyhydroxylated compounds such as polyether diols, polyester diols, polyacetal diols, polyamide diols, polyester polyamide diols, poly(alkylene ether) diols, polythioether diols and polycarbonate diols.

The polyether diols may be chosen, for example, from the products of condensation of ethylene oxide, propylene oxide, or tetrahydrofuran, and their graft or block copolymerization and condensation products such as mixtures of condensates of ethylene and propylene oxide, and the products of polymerization of olefins, at high pressure, with condensates of alkylene oxide. Suitable polyether diols may be prepared, for example, by condensation of alkylene oxides and polyhydric alcohols such as ethylene glycol, 1,2-propylene glycol, and 1,4-butanediol.

In at least one embodiment, the polyester diols, polyester amides, and polyamide diols are saturated and are obtained, for example, from the reaction of saturated or unsaturated polycarboxylic acids with polyhydric alcohols, diamines, or polyamines. To prepare these compounds, it is possible to use, for example, acids chosen from adipic acid, succinic acid, phthalic acid, terephthalic acid, and maleic acid. Suitable polyhydric alcohols for preparing the polyesters include, for example, ethylene glycol, 1,2-propylene glycol, 1,4-butanediol, neopentyl glycol, and hexanediol. Amino alcohols, for example, ethanolamine, may also be used. Suitable diamines for preparing the amide polyesters may be chosen, for instance, from ethylenediamine and hexamethylenediamine.

Suitable polyacetals may be prepared, for example, from 1,4-butanediol or hexanediol and formaldehyde. Suitable polythioethers may be prepared, for example, by a condensation reaction between thioglycols alone or in combination with other glycols such as ethylene glycol and 1,2-propylene glycol or with other polyhydroxylated compounds. Polyhydroxylated compounds already comprising urethane groups, natural polyols, which may optionally be further modified, for example, castor oil and carbohydrates, may also be used.

According to at least one embodiment, the at least one compound of group (1) is a polyesterol, for example, a polyesterdiol formed by the reaction of at least one (di)-polyol (1a) and at least one acid (1b). The at least one (di)-polyol (1a) may be chosen, for example, from neopentyl glycol, 1,4-butanediol, hexanediol, ethylene glycol, diethylene glycol, propylene glycol, butylene glycol, neopentyl glycol, and (di)-polyethylene glycol. The acid (1b) may be chosen, for example, from phthalic acid, isophthalic acid, adipic acid, and (poly)lactic acid.

Non-limiting examples of compounds (2) include hydroxycarboxylic acids such as dimethylolpropanoic acid (DMPA) and 2,2-hydroxymethylcarboxylic acid. In general, compound (2) may be useful as a coupling block. According to one embodiment, the at least one compound (2) may comprise at least one poly(α,α-dihydroxylated carboxylic acid).

In another embodiment, the at least one compound (2) may be chosen from 2,2-di(hydroxymethyl)acetic acid, 2,2-dihydroxymethylpropionic acid, 2,2-dihydroxymethylbutyric acid, and 2,2-dihydroxymethylpentanoic acid.

The at least one di- or polyisocyanate (3) may be chosen, for example, from hexamethylene diisocyanate, isophorone diisocyanate (IDPI), tolylene diisocyanate, diphenylmethane-4,4′-diisocyanate (DPMD), dicyclohexylmethane-4,4′-diisocyanate (DCMD), methylene-di-p-phenyl diisocyanate, methylene bis(4-cyclohexyl isocyanate), toluene diisocyanates, 1,5-naphthalene diisocyanate, 4,4′-diphenylmethane diisocyanate, 2,2′-dimethyl-4,4′-diphenylmethane diisocyanate, 1,3-phenylene diisocyanate, 1,4-phenylene diisocyanate, mixtures of 2,4 and 2,6-toluene diisocyanates, 2,2′-dichloro-4,4′-diisocyanatodiphenylmethane, 2,4-dibromo-1,5-diisocyanatonaphthalene, butane-1,4-diisocyanate, hexane-1,6-diisocyanate, and cyclohexane-1,4-diisocyanate.

According to one embodiment, the at least one non-associative polyurethane may be formed with the aid of at least one additional compound (4), which may serve to extend the chain of the at least one polyurethane. These compounds (4) may be chosen, for instance, from saturated or unsaturated glycols such as ethylene glycol, diethylene glycol, neopentyl glycol, and triethylene glycol; amino alcohols such as ethanolamine, propanolamine, and butanolamine; heterocyclic, aromatic, cycloaliphatic, and aliphatic primary amines; diamines; carboxylic acids such as aliphatic, aromatic, and heterocyclic carboxylic acids such as oxalic, succinic, glutaric, adipic, sebacic, and terephthalic acids; and aminocarboxylic acids. In at least one embodiment, the at least one compound (4) is chosen from aliphatic diols.

The at least one non-associative polyurethane may also be further formed from at least one additional compound (5) having a silicone backbone, such as polysiloxanes, polyalkylsiloxanes, and polyarylsiloxanes, for example, polyethylsiloxanes, polymethylsiloxanes, and polyphenylsiloxanes, optionally comprising graft hydrocarbon chains on the silicon atoms.

In at least one embodiment, the at least one non-associative polyurethane may comprise a basic repeating unit chosen from those of formula (VI):
—O—B—O—CO—NH—R—NH—CO— (VI)

wherein:

B is chosen from divalent C₁ to C₃₀ hydrocarbon groups, which may optionally be substituted with a group comprising at least one functional group chosen from carboxylic acid functional groups and sulphonic acid functional groups, the carboxylic and/or sulphonic acid functional groups being in free form or alternatively partially or completely neutralized with an inorganic or organic base, and

R is chosen from divalent groups chosen from C₁ to C₂₀ aliphatic, C₃ to C₂₀ cycloaliphatic, and C₆ to C₂₀ aromatic hydrocarbon groups, for example, C₁ to C₂₀ alkylene, C₆ to C₂₀ arylene, and C₃ to C₂₀ cycloalkylene groups, and combinations thereof, these groups being substituted or unsubstituted.

In at least one embodiment, the group R is chosen from groups of the following formulae:

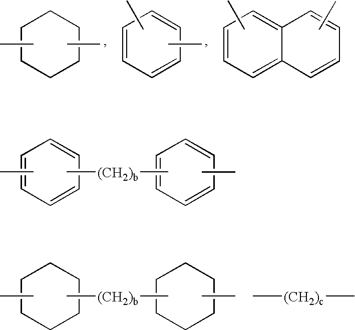

wherein b is an integer ranging from 0 to 3, and c is an integer ranging from 1 to 20, for example, from 2 to 12.

In another embodiment, the group R may be chosen from hexamethylene, 4,4′-biphenylenemethane, 2,4- and/or 2,6-tolylene, 1,5-naphthylene, p-phenylene and methylene-4,4-bis-cyclohexyl groups, and the divalent group derived from isophorone.

The at least one non-associative polyurethane used in accordance with the present disclosure may, in at least one embodiment, additionally comprise at least one polysiloxane block whose basic repeating unit is chosen from those of formula (VII):
—O—P—O—CO—NH—R—NH—CO— (VIl)

wherein:

P is a polysiloxane segment, and

R is a divalent group chosen from C₁ to C₂₀ aliphatic, C₃ to C₂₀ cycloaliphatic, and C₆ to C₂₀ aromatic hydrocarbon groups, for example, C, to C₂₀ alkylene, C₆ to C₂₀ arylene, and C₃ to C₂₀ cycloalkylene groups, and combinations thereof, these groups being substituted or unsubstituted.

According to one embodiment, the polysiloxane segment P is chosen from those of formula (VIII):

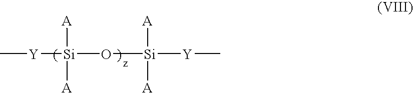

wherein:

the groups A, which may be identical or different, are chosen from, C₁-C₂₀ monovalent hydrocarbon groups free or substantially free of ethylene unsaturation, and aromatic groups,

Y is a divalent hydrocarbon group, and

z is an integer, chosen such that the weight-average molecular mass of the polysiloxane segment ranges from 300 to 10 000.

In another embodiment, the divalent group Y is chosen from alkylene groups of formula —(CH₂)ₐ—, wherein a is an integer ranging from 1 to 10.

The groups A may be chosen, for example, from C₁-C₈ alkyl groups, such as methyl, ethyl, propyl, isopropyl, butyl, pentyl, hexyl, and octyl groups; C₃-C₈ cycloalkyl groups, such as cyclohexyl groups; C₆-C₁₀ aryl groups, such as phenyl; and C₇-C₁₀ arylalkyl groups, such as benzyl, phenylethyl, tolyl, and xylyl groups.

Non-limiting examples of non-associative polyurethanes include the dimethylolpropionic acid/isophorone diisocyanate/neopentyl glycol/polyester diol copolymer (also known by the name polyurethane-1, INCI name) sold under the trademark Luviset® PUR by the company BASF, and the dimethylolpropionic acid/isophorone diisocyanate/neopentyl glycol/polyester diol/silicone diamine copolymer (also known by the name polyurethane-6, INCI name) sold under the trademark Luviset® Si PUR A by the company BASF.

The at least one polyurethane present in the composition according to the present disclosure is used in non-neutralized, and therefore non-ionic, form.

Associative Polyurethanes

As used herein, the expression associative polyurethane is understood to mean a polyurethane possessing at least one terminal or pendant alkyl chain comprising at least 10 carbon atoms. This type of polymer is capable of interacting with itself or with other compounds such as surfactants, leading to a thickening of the medium.

The associative polyurethanes used in the invention are non-ionic.

Suitable non-ionic associative polyurethanes include, but are not limited to, acrylic copolymers which are soluble or capable of swelling in water. Such polyurethanes may comprise:

a) 40 to 99.5% by weight, for example, 30 to 65% by weight, of at least one non-surfactant monoethylenically unsaturated monomer, and

b) 0.5 to 60% by weight, for example, 10 to 50% by weight, of at least one non-ionic urethane monomer which is the product of the reaction of a monohydric non-ionic surfactant with a monoethylenically unsaturated monoisocyanate.

According to at least one embodiment, the copolymer may comprise a large proportion, as indicated above, of at least one ethylenically unsaturated monomer a) which does not have a surfactant property. In another embodiment, the monomers are chosen from those which yield water-insoluble polymers when they are homopolymerized, for example, C₁-C₄ alkyl acrylates and methacrylates such as methyl acrylate, ethyl acrylate, butyl acrylate, and the corresponding methacrylates. According to yet another embodiment, the monomers are chosen from methyl and ethyl (meth)acrylates. Other examples of monomers include, but are not limited to, styrene, vinyltoluene, vinyl acetate, acrylonitrile, and vinylidene chloride. In a further embodiment, the monomers are non-reactive monomers, for example, those in which the single ethylene group is the only reactive group under the polymerization conditions. However, monomers which contain reactive groups under the action of heat may be used in certain situations, such as hydroxyethyl acrylate.

The monohydric non-ionic surfactants used to obtain the non-ionic urethane monomer b) may be chosen from those known in the art, such as alkoxylated hydrophobic compounds comprising an alkylene oxide forming the hydrophilic part of the molecule. The hydrophobic compounds may comprise, for example, an aliphatic alcohol or an alkylphenol in which a carbon chain comprising at least six carbon atoms forms the hydrophobic part of the surfactant.

According to one embodiment, the preferred monohydric non-ionic surfactants may be chosen from those of the following formula:

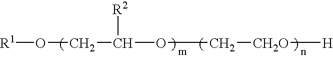

wherein R¹ is chosen from C₆-C₃₀ alkyl and C₈-C₃₀ aralkyl groups, R² is chosen from C₁-C₄ alkyl groups, n is an average number ranging from about 5 to 150 and m is an average number ranging from about 0 to 50, provided that n is at least as big as m and that the sum n+m ranges from 5 to 150.

Examples of C₆-C₃₀ alkyl groups include, but are not limited to, dodecyl and C₁₈-C₂₆ alkyl radicals. Suitable aralkyl groups include, for instance, (C₈-C₁₃)alkylphenyl groups. According to one embodiment, R² group is a methyl group.

The monoethylenically unsaturated monoisocyanate used to form the non-ionic urethane monomer b) may be chosen from a wide variety of compounds known in the art, for example, compounds comprising any copolymerizable unsaturation such as acrylic and methacrylic unsaturation. It is also possible to use an allyl unsaturation conferred by allyl alcohol. In at least one embodiment, the monoethylene monoisocyanates are chosen from α,α-dimethyl-m-isopropenylbenzyl isocyanate and methylstyrene isopropyl isocyanate.

The acrylic copolymer defined above may be obtained by aqueous emulsion copolymerization of the components a) and b) which is described in the art, for example, in European Patent Application No. 0 173 109.

The non-ionic associative polyurethanes used in the present invention may be chosen, for example, from polyurethane polyethers comprising in their chain both hydrophilic blocks, for example, polyoxyethylenated blocks, and hydrophobic blocks, for instance, aliphatic linkages and/or cycloaliphatic and/or aromatic linkages.

In at least one embodiment, the polyurethane polyethers can comprise at least two lipophilic hydrocarbon chains comprising from 6 to 30 carbon atoms, separated by a hydrophilic block, it being possible for the hydrocarbon chains to be pendant chains or chains at the end of a hydrophilic block. In another embodiment, the polyurethane polyethers may comprise at least one pendant lipophilic hydrocarbon chain. In a further embodiment, the polymer may comprise a hydrocarbon chain at one end or at both ends of a hydrophilic block.

The polyurethane polyethers may be multiblock, for example, triblock polymers. The hydrophobic blocks may be at each end of the chain (for example: triblock copolymer with a hydrophilic central block) or distributed both at the ends and in the chain (for example, multiblock copolymers). These polymers may also be chosen from graft and star-shaped polymers.

The non-ionic polyurethane polyethers with a fatty chain may be triblock copolymers whose hydrophilic block is a polyoxyethoxylated chain comprising from 50 to 1000 ethoxylated groups. The non-ionic polyurethane polyethers comprise a urethane bond between the hydrophilic blocks, hence the origin of the name.

By implication, those polymers having hydrophilic blocks linked to the lipophilic blocks by other chemical bonds are also included among the non-ionic polyurethane polyethers with a fatty chain.

Examples of non-ionic polyurethane polyethers with a fatty chain which can be used in accordance with the present disclosure include, but are not limited to, Rheolate 205 with a urea functional group and Rheolates 208, 204, and 212 sold by the company Rheox; Acrysol® RM 184; Elfacos T210 with a C₁₂-C₁₄ alkyl chain and the product and Elfacos® T212 with a C₁₈ alkyl chain, from Akzo; and DW 1206B from Rohm & Haas with a C₂₀ alkyl chain and with a urethane bond, provided at 20% dry matter content in water.

It is also possible to use solutions or dispersions of these polymers, for example, dispersions in water or in an aqueous-alcoholic mediums. Non-limiting examples of such polymers include Rheolate® 255, Rheolate® 278, and Rheolate® 244 sold by the company Rheox and DW 1206F and DW 1206J provided by the company Rohm & Haas.

The polyurethane polyethers which can be used according to the present disclosure may include, for example, those described in G. Fonnum, J. Bakke and Fk. Hansen—Colloid Polym. Sci. 271, p. 380-389 (1993).

Examples of suitable non-ionic associative polyurethanes include, but are not limited to, polyurethane polyethers which may be obtained by polycondensation of at least three compounds comprising (i) at least one polyethylene glycol comprising from 150 to 180 mol of ethylene oxide, (ii) stearyl alcohol or decyl alcohol and (iii) at least one diisocyanate.

Such polyurethane polyethers are sold, for example, by the company Rohm & Haas under the names Aculyn® 46 and Aculyn® 44. Aculyn® 46 is a polycondensate of polyethylene glycol comprising 150 or 180 mol of ethylene oxide, stearyl alcohol, and methylene bis(4-cyclohexyl isocyanate) (SMDI), at 15% by weight in a matrix of maltodextrin (4%) and water (81%); Aculyn® 44 is a polycondensate of polyethylene glycol comprising 150 or 180 mol of ethylene oxide, decyl alcohol and methylene bis(4-cyclohexyl isocyanate) (SMDI), at 35% by weight in a mixture of propylene glycol (39%) and water (26%).

The at least one non-ionic polyurethane may be present in the cosmetic composition in an amount ranging from 0.1% to 99.9% by weight, for example, from 0.1 to 40% by weight, relative to the total weight of the cosmetic composition.

Nucleophilic Agents

The at least one nucleophilic agent capable of initiating the anionic polymerization may be chosen from systems known in the art that are capable of generating a carbanion upon contact with a nucleophilic agent, such as the hydroxide ions contained in water at neutral pH. As used herein, the expression “carbanion” is understood to mean the chemical species defined in “Advanced Organic Chemistry, Third Edition”, by Jerry March, page 141.

The at least one nucleophilic agent may be applied to the hair independently of the composition of the present disclosure. In another embodiment, the at least one nucleophilic agent may be added to the composition of the present disclosure at the time of use.

The at least one nucleophilic agent may be chosen, for example, from molecular compounds, oligomers, dendrimers, and polymers possessing nucleophilic functional groups. Non-limiting examples of such agents include: R₂N⁻, NH₂⁻, Ph₃C⁻, R₃C⁻, PhNH⁻, pyridine, ArS⁻, R—C≡C⁻, RS⁻, SH⁻, RO⁻, R₂NH, ArO⁻, N₃⁻, OH⁻, ArNH₂, NH₃, I⁻, Br⁻, Cl⁻, RCOO⁻, SCN⁻, ROH, RSH, NCO⁻, CN⁻, NO₃⁻, ClO₄⁻, and H₂O functional groups; wherein Ph is a phenyl group; Ar is an aryl group, and R is chosen from C₁-C₁₀ alkyl groups. According to one embodiment, the at least one nucleophilic agent is water.

Pigments

The cosmetic composition of the present disclosure may additionally comprise at least one pigment.

The use of a pigment in the cosmetic composition according to the invention makes it possible to obtain colorations which are visible on dark hair since the pigment at the surface masks the natural color of the fiber.

The composition of the present disclosure may thus have the advantage of producing colorations which exhibit good resistance to various attacks to which the hair may be subjected, such as fatty substances and shampoos.

Furthermore, the cosmetic composition according to the present disclosure may make it possible to produce visible and highly chromatic colorations on a dark keratin fiber without the need to lighten or bleach the keratin fibers and, consequently, without physical degradation of the keratin fibers.

As used herein, the expression “pigment” is understood to mean any organic and/or inorganic entity having a solubility in water is less than 0.01% at 20° C., for example, less than 0.0001%, and exhibiting an absorption ranging from 350 to 700 nm, and in at least one embodiment, exhibiting an absorption with a maximum.

The pigments which may be used in the composition according to the present disclosure may be chosen, for example, from organic and/or inorganic pigments known in the art, such as those described in the Encyclopaedia of Chemical Technology by Kirk-Othmer and in the Encyclopaedia of Industrial Chemistry by Ullmann.

These pigments may be provided in powdered form or pigmented paste form. They may be coated or uncoated.

The pigments used in accordance with the present disclosure may, for example, be chosen from white and colored pigments, lacquers, pigments with special effects such as pearlescent agents and glitter, and mixtures thereof.

Examples of white and colored inorganic pigments include, but are not limited to, optionally surface-treated titanium dioxide, zirconium oxides, cerium oxides, iron oxides, chromium oxides, manganese violet, ultramarine blue, chromium hydrate, and ferric blue. Further non-limiting examples include: Ta₂O₅; Ti₃O₅; Ti₂O₃; TiO; mixtures of ZrO₂ and TiO₂; ZrO₂; Nb₂O₅; CeO₂; and ZnS.

Suitable white and colored organic pigments include, for example, nitroso, nitro, azo, xanthene, quinoline, anthraquinone, phthalocyanine, metal complex type, isoindolinone, isoindoline, quinacridone, perinone, perylene, diketopyrrolopyrrole, thioindigo, dioxazine, triphenylmethane, and quinophthalone compounds.

In at least one embodiment, the white and colored organic pigments may be chosen from carmine; carbon black; aniline black; azo yellow; quinacridone; phthalocyanine blue; sorghum red; the blue pigments codified in the Color Index under the references Cl 42090, 69800, 69825, 73000, 74100, and 74160; the yellow pigments codified in the Color Index under the references Cl 11680, 11710, 15985, 19140, 20040, 21100, 21108, 47000, and 47005; the green pigments codified in the Color Index under the references Cl 61565, 61570, and 74260; the orange pigments codified in the Color Index under the references Cl 11725, 15510, 45370, and 71105; the red pigments codified in the Color Index under the references Cl 12085, 12120, 12370, 12420, 12490, 14700, 15525, 15580, 15620, 15630, 15800, 15850, 15865, 15880, 17200, 26100, 45380, 45410, 58000, 73360, 73915, and 75470; and the pigments obtained by oxidative polymerization of indole or phenol derivatives, such as those described in French Patent No. 2 679 771.

It is also possible to use pigmented pastes of organic pigment such as the products sold by the company Hoechst under the names:

Jaune Cosmenyl IOG: Pigment Yellow 3 (Cl 11710);

Jaune Cosmenyl G: Pigment Yellow 1 (Cl 11680);

Orange Cosmenyl GR: Pigment Orange 43 (Cl 71105);

Rouge Cosmenyl R: Pigment Red 4 (Cl 12085);

Carmin Cosmenyl FB: Pigment Red 5 (Cl 12490);

Violet Cosmenyl RL: Pigment Violet 23 (Cl 51319);

Bleu Cosmenyl A2R: Pigment Blue 15.1 (Cl 74160);

Vert Cosmenyl GG: Pigment Green 7 (Cl 74260); and

Noir Cosmenyl R: Pigment Black 7 (Cl 77266).

The pigments in accordance with the present disclosure may also be in the form of composite pigments, such as those described in European Patent No. 1 184 426. These composite pigments may comprise particles comprising an inorganic core, at least one binder enabling the attachment of the organic pigments to the core, and at least one organic pigment at least partially covering the core.

As used herein, the expression “lacquer” is understood to mean dyes adsorbed onto insoluble particles, the combination thus obtained remaining insoluble during use. The inorganic substrates onto which the dyes are adsorbed may be chosen, for example, from alumina, silica, calcium and sodium borosilicate, calcium and aluminum borosilicate, and aluminum. A non-limiting example of a suitable organic dye is carmine.

Examples of lacquers include, but are not limited to, the following products: D & C Red 21 (Cl 45 380), D & C Orange 5 (Cl 45 370), D & C Red 27 (Cl 45 410), D & C Orange 10 (Cl 45 425), D & C Red 3 (Cl 45 430), D & C Red 7 (Cl 15 850:1), D & C Red 4 (Cl 15 510), D & C Red 33 (Cl 17 200), D & C Yellow 5 (Cl 19 140), D & C Yellow 6 (Cl 15 985), D & C Green (Cl 61 570), D & C Yellow 10 (Cl 77 002), D & C Green 3 (Cl 42 053), and D & C Blue 1 (Cl 42 090).

As used herein, the expression “pigments with special effects” is understood to mean pigments which create in general a colored appearance (characterized by a certain shade, a certain brilliance, and a certain clarity) which is not uniform and changes according to the conditions of observation (e.g., light, temperature, angles of observation, and the like). They are thereby the opposite of white and colored pigments which provide a conventional opaque, semitransparent, or transparent uniform color.

Examples of pigments with special effects include, but are not limited to, white pearlescent pigments such as titanium-coated mica, titanium coated with bismuth oxychloride, colored pearlescent pigments such as mica coated with titanium and iron oxides, mica coated with titanium and ferric blue or chromium oxide, mica coated with titanium and at least one organic pigment as defined above, pearlescent pigments based on bismuth oxychloride, and mica coated with iron oxide. Non-limiting examples of pearlescent pigments include the pearlescent agents Cellini marketed by Engelhard (mica-TiO₂-lacquer), Prestige marketed by Eckart (mica-TiO₂), Colorona marketed by Merck (mica-TiO₂-Fe₂O₃), Prestige bronze marketed by Eckart (mica-Fe₂O₃), and Sunshine Super Copper marketed by Sun Chemicals (mica-Fe₂O₃).

Pigments with interferential effects not attached to a substrate may also be used, such as liquid crystals (Helicones HC from Wacker), and interferential holographic glitter (Geometric Pigments or Spectra f/x from Spectratek). The pigments with special effects may also include fluorescent pigments, for example,substances fluorescent to daylight, substances which produce an ultraviolet fluorescence, phosphorescent pigments, photochromic pigments, thermochromic pigments, and quantum dots, marketed for example by the company Quantum Dots Corporation.

Quantum dots are luminescent semiconducting nanoparticles capable of emitting, under light excitation, a ray having a wavelength ranging from 400 nm to 700 nm. These nanoparticles are known in the art and may be manufactured according to the methods described, for example, in U.S. Pat. Nos. 6,225,198 and 5,990,479, in the publications cited therein, and in the following publications: Dabboussi B. O. et al “(CdSe)ZnS core-shell quantum dots: synthesis and characterisation of a size series of highly luminescent nanocristallites” Journal of physical chemistry B, vol. 101, 1997, p. 9463-9475 and Peng, Xiaogang et al, “Epitaxial Growth of highly Luminescent CdSe/CdS core/shell nanocrystals with photostability and electronic accessibility” Journal of the American Chemical Society, vol. 119, No. 30, p. 7019-7029.

The variety of pigments which may be used in the present invention may make it possible to obtain a rich palette of colors, and optical effects such as interferential metallic effects.

According to one embodiment, the at least one pigment is a colored pigment. As used herein, the expression “colored pigments” is understood to mean pigments other than white pigments.

The size of the at least one pigment useful in the context of the present invention may range from 10 nm to 200 μm, for example, from 20 nm to 80 μm, or from 30 nm to 50 μm.

According to at least one embodiment, the at least one pigment present in the cosmetic composition according to the invention is a pearlescent pigment, for example, mica coated with iron oxide.

In another embodiment, the at least one pigment may be coated with at least one agent chosen from organic and inorganic compounds.

The at least one organic agent with which the pigments may be treated may be deposited on the pigments by solvent evaporation, by chemical reaction between the molecules of the surface-active agent, or by creation of a covalent bond between the surface-active agent and the pigments or the fillers.

The surface treatment may thus be carried out, for example, by the chemical reaction of a surface-active agent with the surface of the pigments and the creation of a covalent bond between the surface-active agent and the pigments, as described, for example, in U.S. Pat. No. 4,578,266.

According to one embodiment, an organic agent covalently linked to the pigments or to the fillers will be used.

The at least one agent for the surface treatment may be present in an amount ranging from 0.1 to 50% by weight of the total weight of the pigments or fillers that are surface-treated, for example, from 0.5 to 30% by weight, or from 1 to 10% by weight.

In at least one embodiment, the at least one pigment may be treated using at least one of the following treatments:

a PEG-silicone treatment such as the AQ surface treatment marketed by LCW;

a Chitosan treatment such as the CTS surface treatment marketed by LCW;

a Triethoxycaprylylsilane treatment such as the AS surface treatment marketed by LCW;

a Methicone treatment such as the SI surface treatment marketed by LCW;

a Dimethicone treatment such as the Covasil 3.05 surface treatment marketed by LCW;

a Dimethicone/Trimethyl siloxysilicate treatment such as the Covasil 4.05 surface treatment marketed by LCW;

a Lauroyl Lysine treatment such as the LL surface treatment marketed by LCW;

a Lauroyl Lysine Dimethicone treatment such as the LUSI surface treatment marketed by LCW;

a Magnesium Myristate treatment such as the MM surface treatment marketed by LCW;

an Aluminum Dimyristate treatment such as the MI surface treatment marketed by Miyoshi;

a Perfluoropolymethylisopropyl ether treatment such as the FHC surface treatment marketed by LCW;

an Isostearyl Sebacate treatment such as the HS surface treatment marketed by Miyoshi;

a Disodium Stearoyl Glutamate treatment such as the NAI surface treatment marketed by Miyoshi;

a Dimethicone/Disodium Stearoyl Glutamate treatment such as the SA/NAI surface treatment marketed by Miyoshi;

a Perfluoroalkyl Phosphate treatment such as the PF surface treatment marketed by Daito;

an Acrylate/Dimethicone and Perfluoroalkyl Phosphate treatment such as the FSA surface treatment marketed by Daito;

a Polymethylhydrogen Siloxane/Perfluoroalkyl Phosphate treatment such as the FS01 surface treatment marketed by Daito;

a Lauryl Lysine/Aluminum Tristearate treatment such as the LL-StAI surface treatment marketed by Daito;

an Octyltriethylsilane treatment such as the OTS surface treatment marketed by Daito;

an Octyltriethylsilane/Perfluoroalkyl Phosphate treatment such as the FOTS surface treatment marketed by Daito;

an Acrylate Dimethicone copolymer treatment such as the ASC surface treatment marketed by Daito;

an Isopropyl Titanium Triisostearate treatment such as the ITT surface treatment marketed by Daito;

a Microcrystalline Cellulose and Carboxymethyl Cellulose treatment such as the AC surface treatment marketed by Daito;

a Cellulose treatment such as the C2 surface treatment marketed by Daito;

an Acrylate Copolymer treatment such as the APD surface treatment marketed by Daito; and

a Perfluoroalkyl Phosphate/Isopropyl Titanium Triisostearate treatment such as the PF+ITT surface treatment marketed by Daito.

The at least one pigment may be present in the composition in an amount,f or each of them, ranging from 0.05 to 50% by weight, for example, from 0.1 to 35% by weight, relative to the total weight of the composition.

Polymerization Inhibitors

The composition of the present disclosure may further comprise at least one polymerization inhibitor, for instance, inhibitors of anionic and/or free-radical polymerization, so as to increase the stability of the composition over time. Non-limiting examples of polymerization inhibitors include: sulphur dioxide, nitric oxide, boron trifluoride, hydroquinone and its derivatives such as hydroquinone monoethyl ether, TBHQ, benzoquinone and its derivatives such as duroquinone, catechol and its derivatives such as t-butyl catechol and methoxycatechol, anisole and its derivatives such as methoxyanisole and hydroxyanisole, pyrogallol and its derivatives, p-methoxyphenol, butylated hydroxytoluene, alkyl sulphates, alkyl sulphites, alkyl sulphones, alkyl sulphoxides, alkyl sulphides, mercaptans, 3-sulphonene and their derivatives. In one embodiment, the alkyl groups may be chosen from groups comprising from 1 to 6 carbon atoms.

Inorganic and organic acids may also be used as polymerization inhibitors.

Thus, the cosmetic composition according to the present disclosure may further comprise at least one acid chosen from inorganic and organic acids, it being possible for the latter to have at least one group chosen from carboxyl and sulphonic groups, having a pKa ranging from 0 to 6, such as phosphoric acid, hydrochloric acid, nitric acid, benzene- or toluenesulphonic acid, sulphuric acid, carbonic acid, hydrofluoric acid, acetic acid, formic acid, propionic acid, benzoic acid, mono-, di-, and trichloroacetic acids, salicylic acid, trifluoroacetic acid, octanoic acid, heptanoic acid, and hexanoic acid.

According to at least one embodiment, the at least one acid is acetic acid.

The at least one polymerization inhibitor may be present in the cosmetic composition in an amount ranging from 10 ppm to 30% by weight, for example, from 10 ppm to 15% by weight, relative to the total weight of the composition. Cosmetically Acceptable Medium

The composition of the present disclosure may comprise a cosmetically acceptable medium chosen from water, at least one liquid organic solvent, and mixtures of water and at least one liquid organic solvent.

As used herein, the expression “organic solvent” is understood to mean an organic substance capable of dissolving another substance without chemically modifying it.

The organic solvents may be chosen from compounds which are liquid at the temperature of 25° C. and at 105 Pa (760 mmHg).

In the context of the present disclosure, the at least one cyanoacrylate monomer and the at least one organic solvent are distinct compounds.

The at least one organic solvent may be chosen, for example, from aromatic alcohols such as benzyl alcohol; liquid fatty alcohols, such as C₁₀-C₃₀ alcohols; modified or unmodified polyols such as glycerol, glycol, propylene glycol, dipropylene glycol, butylene glycol, and butyl diglycol; volatile silicones such as cyclopentasiloxane, cyclohexasiloxane, polydimethylsiloxanes which are optionally modified with at least one functional group chosen from alkyl, amine, imine, fluoroalkyl, carboxyl, betaine, and quaternary ammonium functional groups, and modified liquid polydimethylsiloxanes; mineral, organic, and vegetable oils; alkanes, such as C₅ to C₁₀ alkanes; liquid fatty acids; and liquid fatty esters, such as liquid fatty alcohol benzoates and salicylates.

The at least one organic solvent may be chosen, for example, from organic oils; silicones such as volatile silicones, amino or non-amino silicone gums and oils, and mixtures thereof; mineral oils; vegetable oils such as olive, castor, rapeseed, coconut, wheat germ, sweet almond, avocado, macadamia, apricot, safflower, candlenut, camelina, tamanu, and lemon oils; and organic compounds such as C₅-C₁₀ alkanes, acetone, methyl ethyl ketone, esters of liquid C₁-C₂₀ acids and of C₁-C₈ alcohols such as methyl acetate, butyl acetate, ethyl acetate, and isopropyl myristate, dimethoxyethane, diethoxyethane, liquid C₁₀-C₃₀ fatty alcohols such as oleyl alcohol, liquid C₁₀-C₃₀ fatty alcohol esters such as C₁₀-C₃₀ fatty alcohol benzoates, and mixtures thereof; polybutene oil; isononyl isononanoate; isostearyl malate; pentaerythrityl tetraisostearate; tridecyl trimelate; the mixture cyclopentasiloxane (14.7% by weight)/polydimethylsiloxane dihydroxylated at the α and γ positions (85.3% by weight); and mixtures thereof.

According to one embodiment, the at least one organic solvent is chosen from silicones and silicone mixtures such as liquid polydimethylsiloxanes and modified liquid polydimethylsiloxanes, wherein the viscosity of the silicone and/or of the silicone mixture at 25° C ranges from 0.1 cst and 1 000 000 cst, for example, from 1 cst to 30 000 cst.

Non-limiting examples of suitable organic solvents include the following oils and oil mixtures:

the mixture of alpha-omega-dihydroxylated polydimethylsiloxane/cyclopentadimethylsiloxane (14.7/85.3) marketed by Dow Corning under the name DC 1501 Fluid,

the mixture of alpha-omega-dihydroxylated polydimethylsiloxane/polydimethylsiloxane marketed by Dow Corning under the name DC 1503 Fluid,

the mixture of dimethicone/cyclopentadimethylsiloxane marketed by Dow Corning under the name DC 1411 Fluid or that marketed by Bayer under the name SF1214;

the cyclopentadimethylsiloxane marketed by Dow Corning under the name DC 245 Fluid; and

mixtures of these oils.

The composition of the present disclosure may comprise water in addition to the at least one liquid organic solvent. However, according to one embodiment, the composition of the present disclsoure is anhydrous, i.e., comprising less than 1% by weight of water relative to the total weight of the composition.

The cosmetically acceptable medium chosen from water, organic solvents, and mixtures of water and organic solvents may be present in the composition in an amount ranging from 0.01 to 99% by weight, for example, from 50 to 99% by weight relative to the total weight of the composition.

The cosmetically acceptable medium may also be provided in the form of an emulsion and/or may be encapsulated, the at least one cyanoacrylate monomer being maintained in an anhydrous medium up to the time of use. When the medium is an emulsion, this emulsion may comprise a dispersed or continuous phase which may comprise a medium chosen from water, C₁-C₄ aliphatic alcohols, and mixtures thereof, and an anhydrous organic phase comprising the at least one cyanoacrylate monomer. In the case of capsules or microcapsules, the capsule may contain the at least one cyanoacrylate monomer in an anhydrous medium and may be dispersed in a medium chosen from anhydrous media as defined above, water, C₁-C₄ aliphatic alcohols, and mixtures thereof.

Cosmetic Additives

The cosmetic composition of the present disclosure may further comprise at least one cosmetic additive chosen from customary cosmetic additives, such as reducing agents, oxidizing agents, sequestrants, polymeric and nonpolymeric thickening agents, moisturizing agents, emollients, organic and inorganic bases, plasticizers, sunscreens, optical brighteners, oxidation dyes, inorganic fillers, clays, colloidal minerals, colloidal metals, particles of semiconductors of the “quantum well” type based on metals or silicon, photo- or thermochromic compounds, pearlescent agents, perfumes, gum inhibitors, preservatives, proteins, vitamins, antidandruff agents, fixing and non-fixing anionic, cationic, and amphoteric polymers, and nonpolymeric conditioners such as cationic surfactants.

Formulations

The formulations may be provided in various galenic forms such as lotions, aerosol mousses, after-shampoos, shampoos, gels, and waxes. The compositions may be packaged in containers chosen from pump dispensers and aerosol sprays. The compositions of the present disclosure may or may not be rinsed off after application to the keratin fibers.

When the composition is contained in an aerosol, it may further comprise at least one propellant. The at least one propellant may be chosen from compressed and liquefied gases conventionally used for the preparation of aerosol compositions, for example, air, carbon monoxide, compressed nitrogen, soluble gases such as dimethyl ether, halogenated (e.g., fluorinated) hydrocarbons and non-halogenated hydrocarbons (e.g., butane, propane, and isobutane), and mixtures thereof.

According to the method of the invention, the composition of the invention is applied to keratin fibers, in particular human keratin fibers such as the hair, in the presence of a nucleophilic agent.

Methods

Disclosed herein is also a method for treating keratin fibers, for example, for dyeing and/or conditioning keratin fibers, comprising applying to the fibers at least one composition of the present disclosure.

According to one embodiment, the at least one nucleophilic agent capable of initiating the polymerization of the at least one cyanoacrylate monomer may be applied to the keratin fibers before application of the composition of the present disclosure. The at least one nucleophilic agent may be used pure, in solution, in the form of an emulsion, or may be encapsulated. It may also be added to the anhydrous composition at the time of use just before application to the keratin fibers.

According to another embodiment, the at least one nucleophilic agent is water. This water may be provided, for example, by prior wetting of the keratin fibers, or it may be added directly in the composition before application.

According to a further embodiment, it is possible to modulate the kinetics of polymerization by wetting the fiber beforehand with the aid of an aqueous solution whose pH has been adjusted with the aid of an agent chosen from bases, acids, and acid/base mixtures. The acid and/or base may be inorganic or organic.

According to another embodiment, when the at least one non-ionic polyurethane is provided in the form of an aqueous or ethanolic solution or dispersion, this polymer may be applied to the keratin fibers in the form of a pretreatment.

According to yet another embodiment, the method for treating the keratin fibers may be a dyeing method, when the composition comprises at least one pigment. According to this embodiment, the composition of the present disclosure may comprise the at least one pigment, or the hair dyeing may be carried out in several steps. For example, the method may comprise applying a composition comprising the at least one pigment to the fibers and then applying a composition according to the present disclosure comprising, inter alia, the at least one cyanoacrylate monomer, wherein the at least one nucleophilic agent may be present in the composition comprising the at least one pigment or it may be present in a separate composition.

According to this embodiment, the cosmetic composition containing the at least one pigment may be an aqueous solution of pigments, which allows wetting of the fiber and the initiation of the polymerization when the composition according to the present disclosure is applied.

The methods of the present disclosure may comprise at least one additional intermediate or final step, such as applying a cosmetic product, rinsing, and/or drying. The drying may be carried out under a hood, using a hair dryer, and/or using a smoothing iron. In at least one embodiment, the application of the compositions in accordance with the present disclosure may be followed by rinsing.

It is also possible to carry out multiple applications of the composition of the present disclosure in order to obtain superposition of layers in order to achieve specific properties of the deposit in terms of chemical nature, mechanical resistance, thickness, appearance, and/or feel.

In order to improve, inter alia, the adhesion of the poly(cyanoacrylate) formed in situ, the fiber may be pretreated with at least one polymer chosen from any type of polymers.

To modulate the kinetics of anionic polymerization, it is also possible to increase the nucleophilicity of the fiber by chemical transformation of the keratin fibers. For example, the disulphide bridges partly constituting the keratin to thiols may be reduced before application of the composition of the present disclosure. Non-limiting examples of reducing agents for the disulphide bridges partly constituting keratin include: anhydrous sodium thiosulphate, powdered sodium metabisulphite, thiourea, ammonium sulphite, thioglycolic acid, thiolactic acid, ammonium thiolactate, glyceryl monothioglycolate, ammonium thioglycolate, thioglycerol, 2,5-dihydroxybenzoic acid, diammonium dithioglycolate, strontium thioglycolate, calcium thioglycolate, zinc formosulphoxylate, isooctyl thioglycolate, dl-cysteine, and monoethanolamine thioglycolate.

Also disclosed herein is a method for conditioning keratin fibers comprising applying to the fibers at least one composition of the present disclosure.

In at least one embodiment, when the cosmetic composition comprises at least one pigment, the cosmetic composition may be used for conditioning and/or dyeing keratin fibers, such as the hair.

Multi-Compartment Kits

Further disclosed herein is a multi-compartment device or kit, comprising at least one first compartment comprising a composition according to the present disclosure, and at least one second compartment, comprising at least one nucleophilic agent.

Still further disclosed herein is a multi-compartment device or kit, comprising at least one first compartment, comprising at least one non-ionic polyurethane, a second compartment comprising at least one cyanoacrylate monomer, wherein the at least one nucleophilic agent is optionally present in the first and/or second compartment or in a third compartment, it being optionally possible for each compartment to contain a liquid organic solvent.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, unless otherwise indicated the numerical values set forth in the specific examples are reported as precisely as possible. Any numerical value, however, inherently contains certain errors necessarily resulting from the standard deviation found in their respective testing measurements.

By way of non-limiting illustration, concrete examples of certain embodiments of the present disclosure are given below.

### EXAMPLES

### Example 1

The following composition A was prepared:

| | |
|---|---|
| ACULYN 44* from ROHM and HAAS | 10 g |
| Water | 90 g |

*Solution of polyurethane at 35% in a mixture of water and propylene glycol

The following composition B was prepared:

| | |
|---|---|
| DC 1501 Fluid | 20 g |
| DC 245 Fluid | 44.75 g |
| Methylheptyl cyanoacrylate | 10 g |
| marketed by Chemence | |
| Acetic acid | 0.25 g |

0.5 g of composition A was applied to a lock of 1 g of clean and wet hair. The lock may be optionally dried at this stage. 0.5 g of composition B was then applied. After leaving in for 15 minutes, the lock was dried with a hair dryer for 2 minutes. The coatings obtained were stable and thick, which gave a sensation of coating or mass which persisted after shampooing.

### Example 2

### Colored Coating

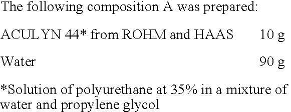

The following composition B′ was prepared:

| | |
|---|---|
| DC 1501 Fluid | 20 g |
| DC 245 Fluid | 44.75 g |
| Pearlescent mica coated with brown iron oxide | 10 g |
| marketed by Eckart under Prestige Bronze | |
| Methylheptyl cyanoacrylate | 10 g |
| marketed by Chemence | |
| Acetic acid | 0.25 g |

0.5 g of composition A was applied to a lock of 1 g of clean and wet hair. The lock may be optionally dried at this stage. 0.5 g of composition B′ was then applied. After leaving in for 15 minutes, the lock was dried with a hair dryer for 2 minutes. The coatings obtained were stable and thick, which gave a sensation of coating or mass which persisted after shampooing.

What is claimed is:

1. A cosmetic product for treating keratin fibers, comprising:
at least one polymerizable cyanoacrylate monomer, and
at least one non-ionic polyurethane,
wherein the at least one polymerizable cyanoacrylate monomer and the at least one non-ionic polyurethane are present in the same composition or in separate form, and may, at the time of use, be applied together or applied separately, simultaneously or sequentially over time.

2. The cosmetic product of claim 1, comprising, in the same composition:
at least one polymerizable cyanoacrylate monomer, and
at least one non-ionic polyurethane.

3. The cosmetic composition of claim 2, wherein the at least one cyanoacrylate monomer is chosen from monomers of formula (I): 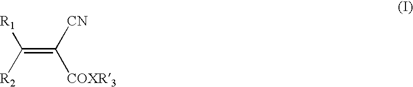
wherein:
X is chosen from NH, S, and O,
R₁ and R₂, which may be identical or different, are weakly or non-electron-attracting (weakly or non-inductive-attracting) groups chosen from:
hydrogen,
saturated or unsaturated, linear, branched, or cyclic hydrocarbon groups optionally comprising at least one atom chosen from nitrogen, oxygen, and sulphur atoms, and optionally substituted with at least one group chosen from —OR, —COOR, —COR, —SH, —SR, —OH, and halogen atoms,
modified or unmodified polyorganosiloxane residues, and
polyoxyalkylene groups,
R is chosen from saturated or unsaturated linear, branched, or cyclic hydrocarbon groups optionally comprising at least one atom chosen from nitrogen, oxygen, and sulphur atoms, and optionally substituted with at least one group chosen from —OR′, —COOR′, —COR′, —SH, —SR′, —OH, halogen atoms, and polymer residues obtained by free-radical polymerization, by polycondensation, or by ring opening, wherein R′ is chosen from C₁-C₁₀ alkyl groups, and
R′₃ is chosen from hydrogen and saturated or unsaturated, linear, branched, or cyclic hydrocarbon groups optionally comprising at least one atom chosen from nitrogen, oxygen, and sulphur atoms, and optionally substituted with at least one group chosen from —OR′, —COOR′, —COR′, —SH, —SR′, —OH, halogen atoms, and polymer residues obtained by free-radical polymerization, by polycondensation, or by ring opening, wherein R′ is chosen from C₁-C₁₀ alkyl groups.

4. The cosmetic composition of claim 3, wherein the at least one cyanoacrylate monomer is chosen from monomers of formula (IV): 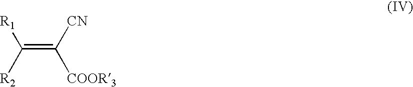
wherein R′₃ is chosen from C₁-C₁₀ alkyl radicals and (C₁-C₄)alkoxy(C₁-C₁₀)alkyl radicals, and R₁ and R₂ are as defined in claim 3.

5. The cosmetic composition of claim 4, wherein the at least one cyanoacrylate monomer is chosen from ethyl 2-cyanoacrylate, methyl 2-cyanoacrylate, n-propyl 2-cyanoacrylate, isopropyl 2-cyanoacrylate, tert-butyl 2-cyanoacrylate, n-butyl 2-cyanoacrylate, isobutyl 2-cyanoacrylate, 3-methoxybutyl cyanoacrylate, n-decyl cyanoacrylate, hexyl 2-cyanoacrylate, 2-ethoxyethyl 2-cyanoacrylate, allyl cyanoacrylate, 2-methoxypropyl cyanoacrylate, 2-methoxyethyl 2-cyanoacrylate, 2-methylheptyl 2-cyanoacrylate, 2-propoxyethyl 2-cyanoacrylate, n-octyl 2-cyanoacrylate, and isoamyl cyanoacrylate.

6. The cosmetic composition of claim 5, wherein the at least one cyanoacrylate monomer is chosen from C₆-C₁₀ alkyl cyanoacrylates.

7. The cosmetic composition of claim 6, wherein the at least one cyanoacrylate monomer is chosen from octyl cyanoacrylates of formula (V) and mixtures thereof: 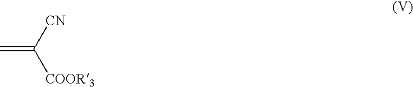
wherein R′₃ is chosen from:
—(CH₂)₇—CH₃,
—CH(CH₃)—(CH₂)₅—CH₃,
—CH₂—CH(C₂H₅)—(CH₂)₃—CH₃,
—(CH₂)₅—CH(CH₃)—CH₃, and
—(CH₂)₄—CH(C₂H₅)—CH₃.

8. The cosmetic composition of claim 1, wherein the at least one cyanoacrylate monomer is covalently linked to at least one support chosen from polymers, oligomers, and dendrimers.

9. The cosmetic composition of claim 2, wherein the at least one cyanoacrylate monomer is present in the composition in an amount ranging from 0.1 to 99.9% by weight relative to the total weight of the composition.

10. The cosmetic composition of claim 2, wherein the at least one non-ionic polyurethane is associative.

11. The cosmetic composition of claim 2, wherein the at least one non-ionic polyurethane is non-associative.

12. The composition of claim 11, wherein the at least one non-associative polyurethane comprises a basic repeating unit chosen from units of formula (VI):
—O—B—O—CO—NH—R—NH—CO— (VI)
wherein:
B is a divalent C₁ to C₃₀ hydrocarbon group, which may be optionally substituted with a group comprising at least one functional group chosen from carboxylic acid functional groups and sulphonic acid functional groups, wherein the at least one functional group is in free form or partially or completely neutralized with an inorganic or organic base, and
R is an optionally substituted divalent group chosen from C₁ to C₂₀ aliphatic, C₃ to C₂₀ cycloaliphatic, and C₆ to C₂₀ aromatic hydrocarbon groups and mixtures thereof.

13. The cosmetic composition of claim 12, wherein the at least one non-associative polyurethane is a dimethylolpropionic acid/isophorone diisocyanate/neopentyl glycol/polyester diol copolymer.

14. The cosmetic composition of claim 11, wherein the at least one non-associative polyurethane further comprises a basic repeating unit chosen from those of formula (VII):
—O—P—O—CO—NH—R—NH—CO— (VII)
wherein:
P is a polysiloxane segment, and
R is an optionally substituted divalent group chosen from C₁ to C₂₀ aliphatic, C₃ to C₂₀ cycloaliphatic, and C₆ to C₂₀ aromatic hydrocarbon groups and mixtures thereof.

15. The cosmetic composition of claim 14, wherein the at least one non-associative polyurethane is a dimethylolpropionic acid/isophorone diisocyanate/neopentyl glycol/polyester diol/silicone diamine copolymer.

16. The cosmetic composition of claim 10, wherein the at least one associative polyurethane is an acrylic copolymer comprising:
a) from 40 to 99.5% by weight of a non-surfactant monoethylenically unsaturated monomer, and
b) from 0.5 to 60% by weight of a non-ionic urethane monomer which is the product of the reaction of a monohydric non-ionic surfactant with a monoethylenically unsaturated monoisocyanate.

17. The cosmetic composition of claim 10, wherein the at least one non-ionic associative polyurethane is a polyurethane polyether obtained by polycondensation of at least three compounds comprising (i) at least one polyethylene glycol comprising from 150 to 180 mol of ethylene oxide, (ii) stearyl alcohol or decyl alcohol, and (iii) at least one diisocyanate.

18. The cosmetic composition of claim 17, wherein the at least one non-ionic associative polyurethane is chosen from polycondensates of polyethylene glycol comprising 150 or 180 mol of ethylene oxide, stearyl alcohol, and methylene bis(4-cyclohexyl isocyanate) and polycondensates of polyethylene glycol containing 150 or 180 mol of ethylene oxide, decyl alcohol, and methylene bis(4-cyclohexyl isocyanate).

19. The cosmetic composition of claim 2, wherein the at least one non-ionic polyurethane is present in the composition in an amount ranging from 0.1 and 99.9% by weight relative to the total weight of the composition.

20. The cosmetic composition of claim 1, further comprising at least one pigment.

21. The cosmetic composition of claim 20, wherein the at least one pigment is in a form chosen from powdered form and pigmented paste form.

22. The cosmetic composition of claim 20, wherein the at least one pigment is a mineral pigment chosen from treated or surface-treated titanium dioxide, zirconium oxides, cerium oxides, iron oxides, chromium oxides, manganese violet, ultramarine blue, chromium hydrate, and ferric blue.

23. The cosmetic composition of claim 20, wherein the at least one pigment is an organic pigment chosen from nitroso, nitro, azo, xanthene, quinoline, anthraquinone, phthalocyanine, metal complex type, isoindolinone, isoindoline, quinacridone, perinone, perylene, diketopyrrolopyrrole, thioindigo, dioxazine, triphenylmethane, and quinophthalone compounds.

24. The cosmetic composition of claim 20, wherein the at least one pigment is a composite pigment comprising particles comprising an inorganic core, at least one binder enabling the attachment of the organic pigments to the core, and at least one organic pigment at least partially covering the core.

25. The cosmetic composition of claim 20, wherein the at least one pigment is a lacquer comprising an inorganic substrate chosen from alumina, silica, calcium and sodium borosilicate, calcium and aluminum borosilicate, aluminum, and aluminum onto which at least one dye is adsorbed.

26. The cosmetic composition of claim 20, wherein the at least one pigment is a pigment with special effects chosen from pearlescent pigments, pigments with interferential effects not attached to a substrate, photochromic pigments, thermochromic pigments, and quantum dots.

27. The cosmetic composition of claim 26, wherein the at least one pearlescent pigment is chosen from mica coated with titanium, mica coated with bismuth oxychloride, mica coated with titanium and iron oxides, mica coated with iron oxide, mica coated with titanium and ferric blue or chromium oxide, mica coated with titanium and at least one organic pigment, and pearlescent pigments based on bismuth oxychloride.

28. The cosmetic composition of claim 27, wherein the at least one pearlescent pigment is mica coated with iron oxide.

29. The cosmetic composition of claim 20, wherein the at least one pigment is present in the composition in an amount ranging from 0.05 to 50% by weight relative to the total weight of the composition.

30. The cosmetic composition of claim 29, wherein the at least one pigment is present in the composition in an amount ranging from 0.1 to 35% by weight relative to the total weight of the composition.

31. The cosmetic composition of claim 2, further comprising at least one polymerization inhibitor.

32. The cosmetic composition of claim 31, wherein the at least one polymerization inhibitor is chosen from inorganic and organic acids.

33. The cosmetic composition of claim 32, wherein the at least one polymerization inhibitor is chosen from phosphoric acid, hydrochloric acid, nitric acid, benzene- or toluenesulphonic acid, sulphuric acid, carbonic acid, hydrofluoric acid, acetic acid, formic acid, propionic acid, benzoic acid, mono-, di-, and trichloroacetic acids, salicylic acid, trifluoroacetic acid, octanoic acid, heptanoic acid, and hexanoic acid.

34. The cosmetic composition of claim 33, wherein the at least one polymerization inhibitor is acetic acid.

35. The cosmetic composition of claim 31, wherein the at least one polymerization inhibitor is present in the composition in an amount ranging from 10 ppm to 30% by weight relative to the total weight of the composition.

36. The cosmetic composition of claim 35, wherein the at least one polymerization inhibitor is present in the composition in an amount ranging from 10 ppm to 15% by weight relative to the total weight of the composition.

37. The cosmetic composition of claim 2, wherein the composition is anhydrous.

38. The cosmetic composition of claim 37, further comprising at least one liquid organic solvent chosen from aromatic alcohols, fatty alcohols, modified or unmodified polyols, volatile or non-volatile silicones, mineral, organic, and vegetable oils, oxyethylenated or non-oxyethylenated waxes, paraffins, alkanes, fatty acids, fatty amides, and fatty esters.

39. The cosmetic composition of claim 38, wherein the at least one organic solvent is cyclopentadimethylsiloxane.

40. The cosmetic composition of claim 2, further comprising at least one cosmetic additive chosen from reducing agents, oxidizing agents, sequestrants, polymeric or nonpolymeric thickening agents, moisturizing agents, emollients, organic or inorganic bases, plasticizers, sunscreens, optical brighteners, oxidation dyes, inorganic fillers, clays, colloidal minerals, colloidal metals, nanoparticles of semiconductors of the quantum well type based on metals or silicon, photo- or thermochromic compounds, perfumes, gum inhibitors, preservatives, proteins, vitamins, antidandruff agents, fixing or non-fixing anionic, cationic, and amphoteric polymers, and nonpolymeric conditioners.

41. The cosmetic composition of claim 2, further comprising at least one nucleophilic agent.

42. The cosmetic composition of claim 41, wherein the at least one nucleophilic agent is water.

43. A method for the cosmetic treatment of artificially colored keratin fibers comprising applying at least one cosmetic composition to the fibers in the presence of at least one nucleophilic agent, wherein the cosmetic composition comprises at least one polymerizable cyanoacrylate monomer and at least one non-ionic polyurethane;
wherein the at least one nucleophilic agent is mixed at the time of use in the at least one cosmetic composition or is applied separately.

44. A method for the cosmetic treatment of artificially colored keratin fibers comprising
applying to said fibers a first composition comprising at least one non-ionic polyurethane, and
then applying to the fibers a second composition comprising at least one cyanoacrylate monomer,
wherein the first and/or second composition may further comprise at least one nucleophilic agent.

45. A method for dyeing keratin fibers, comprising
applying to the fibers a first composition containing at least one pigment,
applying to the fibers a second cosmetic composition comprising at least one polymerizable cyanoacrylate monomer and at least one non-ionic polyurethane, and
applying to the fibers at least one nucleophilic agent, wherein the at least one nucleophilic agent may be being present in the first composition or in a separate third composition.

46. The method of claim 45, wherein the first composition is an aqueous composition of pigments and the second composition is anhydrous.

47. A method for conditioning artificially colored keratin fibers comprising applying to the fibers, in the presence of at least one nucleophilic agent, at least one cosmetic composition comprising at least one polymerizable cyanoacrylate monomer and at least one non-ionic polyurethane;
wherein the at least one nucleophilic agent is mixed at the time of use in the at least one cosmetic composition or is applied separately.

48. A multi-compartment device or kit, comprising at least one first compartment, comprising a composition comprising at least one polymerizable cyanoacrylate monomer and at least one non-ionic polyurethane, and at least one second compartment, comprising at least one nucleophilic agent.

49. A multi-compartment device or kit, comprising at least one first compartment, comprising at least one non-ionic polyurethane, and a second compartment comprising at least one cyanoacrylate monomer, wherein the first and/or second compartment may optionally further comprise at least one nucleophilic agent or the at least nucleophilic agent may be present in at least one third compartment, and wherein each compartment may optionally further comprise at least one liquid organic solvent.
